(19) United States
(12) Patent Application Publication
(10) Pub. No.: US 2005/0101534 A1
(43) Pub. Date: May 12, 2005

(54) Compositions and methods for treating or preventing hearing impairment

(75) Inventors: Charles M. Zepp, Hardwick, MA (US); Donald L. Heefner, Waynesboro, PA (US); Paul Rubin, Sudbury, MA (US); Mark G. Currie, Sterling, MA (US)

Correspondence Address: FISH & NEAVE IP GROUP;ROPES & GRAY LLP, ONE INTERNATIONAL PLACE, BOSTON, MA, 02110-2624, US

(73) Assignee: Sepracor, Inc., Marlborough, MA (US)

(21) Appl. No.: 10/712,849

(22) Filed: Nov. 13, 2003

Related U.S. Application Data
(60) Provisional application No. 60/425,878, filed on Nov. 13, 2002.

(51) Int. Cl. A61K 38/17

(52) U.S. Cl. 514/12

(57) ABSTRACT

Ototoxic side-effects have either limited the application of an important group of antibiotics, aminoglycoside antibiotics, or have added further burden to those who have to resort to platinum-containing antineoplastic agents to treat life-threatening tumors. The present invention discloses pharmaceutical compositions that prevent or treat the ototoxic side-effects of aminoglycoside antibiotics and platinum-containing antineoplastic agents. The otoprotective compositions disclosed herein can also be used to treat noise induced hearing loss.

### CROSS-REFERENCE TO RELATED APPLICATIONS

The present application claims benefit of U.S. Provisional Application No. 60/425,878, filed Nov. 13, 2002, the specification of which is hereby incorporated by reference in its entirety.

### BACKGROUND OF THE INVENTION

Hearing loss afflicts over ten percent of the population of the United States. Hearing loss is associated with loss of hair cells of the organ of Corti in the inner ear and is also often accompanied by deterioration of the spiral ganglion neurons which transduce auditory signals to the brain from the hair cells. Agents causing hearing impairment include loud noise, aging, and chemicals including but not limited to aminoglycoside antibiotics and platinum-containing antineoplastic agents, such as cisplatin. In chemically induced hearing impairment, ototoxic agents such as cisplatin and aminoglycoside antibiotics are known to accumulate in cochlear hair cells. Cellular damage to these cells resulting from the accumulation is thought to be the primary reason for hearing impairment.

One hypothesis to account for hearing impairment due to loud noise, age, or chemicals points to reactive oxygen species (ROS) as being the causative agents for cochlear hair cell damage. Some free radical scavengers, iron chelators, and certain NMDA receptor antagonists have been shown to be effective in protecting cochlear hair cells from chemically induced or noise-induced cell death. Past approaches to treat hearing impairment due to noise induced hearing loss (NIHL) or chemically induced hearing loss (CIHL) have included treatment with antioxidants such as aspirin, reduced glutathione, N-methyl-(D)-glucaminedithiocarbamate, (D)-methionine, and iron chelators such as tartrate and maleate. While these compounds have shown efficacy in some animal models of noise or chemically induced hearing loss, to date, only D-methionine has been approved for use to prevent or treat hearing impairment. However, the pharmacological profile of (D)-methionine makes it difficult to administer it to patients.

There is a pressing need for otoprotective compounds that prevent, reduce, or otherwise treat hearing impairment due to noise, age or chemicals. These otoprotective compounds would be useful in the context of hazards posed by loud noises in certain occupational or recreational activities or injuries arising from ototoxic chemicals including counteracting ototoxic side-effects associated with certain chemotherapeutic regimes, or improving quality of life in aging populations experiencing progressive hearing impairment. For instance, ototoxicity of aminoglycosides has limited the applications of this very important group of antibiotics and the ototoxicity of cisplatin adds a further burden to those already facing a life-threatening disease. Thus there is a need for otoprotective compounds that prevent, reduce, or otherwise treat the ototoxic side-effects of aminoglycoside antibiotics or platinum-containing antineoplastic agents, while substantially preserving the in vivo microcidal or anti-tumor properties of these compounds when administered prior to, simultaneously with, or subsequent to administration of such chemotherapeutic drugs.

### SUMMARY OF THE INVENTION

The present invention relates to the use of otoprotective agents to prevent, reduce, or otherwise treat hearing impairment due to NIHL, aging, or CIHL. Of particular interest in the CIHL category are chemotherapeutic drugs such as aminoglycoside antibiotics, platinum-containing antineoplastic agents such as cisplatin, certain quinine-like compounds, and ototoxic diuretic drugs such as the more popular and commonly used loop-diuretics. More specifically, the present invention relates to the use of 2-thio-nitrogen-containing compounds to prevent, reduce, or otherwise treat ototoxicity associated with NIHL, aging, or CIHL, wherein in the case of CIHL due to chemotherapeutic agents the treatment does not compromise the efficacy of chemotherapeutic agents. While the efficacy of 2-thio-nitrogen-containing compounds disclosed herein may be due to their antioxidative properties vis-à-vis reactive oxygen species generated by, for instance, an aminoglycoside antibiotic or a platinum-containing antineoplastic agent, the efficacy may also be due to another mechanism, such as inhibition of nitric oxide synthetase by the otoprotective compounds disclosed in the present invention.

Accordingly, one aspect of the present invention describes a method for preventing, reducing or otherwise treating NIHL, CIHL, or hearing impairment due to aging by administering to a patient a pharmaceutical dosage of an otoprotective agent selected from the structures depicted in the formulae disclosed herein, or a pharmaceutically acceptable salt, solvate, clathrate, prodrug, tautomer or a metabolic derivative thereof.

Furthermore, an improvement in the present invention relates to methods for augmenting treatments which require administration of a chemotherapeutic agent that has an ototoxic, hearing-impairing side effect. The improvement includes administering prophylacticaly or therapeutically an effective amount of an otoprotective agent to prevent, reduce, or treat the ototoxic side effects of the chemotherapeutic drug without impairing its efficacy. The otoprotective agent and chemotherapeutic agent may be provided in various modes including administration prior to, simultaneously with, or subsequent to administration of said ototoxic chemotherapeutic agent. The otoprotective agent and chemotherapeutic agent may also be provided in various forms including but not limited to a single pharmaceutical preparation, e.g. as a single dosage form, or a kit in which each is provided in separate dosages, along with instructions for co-administering the two agents.

The present invention also relates to methods for conducting pharmaceutical business comprising manufacturing, testing, marketing, distributing, and licensing preparations or kits for co-administering an otoprotective agent with an ototoxic chemotherapeutic agent.

### BRIEF DESCRIPTION OF THE FIGURES

FIGS. 1-3 depict graphs of experimental results showing the effectiveness of compounds of the invention in treating or preventing hearing loss at various frequencies.

### DETAILED DESCRIPTION OF THE INVENTION

A. Overview

The present invention discloses compositions and methods for preventing, reducing or treating auditory conditions. In particular, the present invention discloses methods for preventing, reducing, or treating hearing impairment due to loud noise or chemicals and aging.

B. Definitions

The term “CIHL” is an acronym for chemically induced hearing loss. CIHL describes hearing loss due to exposure to certain chemical compounds which destroy cochlear hair cells, thereby causing hearing loss or hearing impairment. Examples of such chemicals include aminoglycoside antibiotics and platinum containing chemotherapeutic agents. The hearing loss may affect both ears or may affect one ear more than the other.

The term “NIHL” is an acronym for noise induced hearing loss. NIHL describes a chronic hearing impairing disease process that occurs gradually over many years of exposure to less intense noise levels, wherein the damage is to the inner ear, and in specific, the cochlea. This type of hearing loss is generally caused by chronic exposure to high intensity continuous noise with superimposed episodic impact or impulse noise. Both an intense sound presented to the ear for a short period of time and a less intense sound that is presented for a longer time period may produce similar damage to the inner ear. The majority of chronic NIHL is due to occupational or industrial exposure. However, a non-occupational form of NIHL also called socioacusis, may result from gunfire, loud music—via concerts or headphones, open vehicles such as motorcycles, snowmobiles or tractors, and power tools to name just a few. Although, the hearing damage is often symmetrical, i.e., both ears are affected, but there are cases, such as hearing loss due to frequent target shooting, which result in asymmetric hearing loss.

The term “hearing loss” refers both to a complete loss of hearing due to noise, chemicals, or age, or to a hearing impairment due to the aforementioned factors. The term “hearing impairment” refers to a diminished hearing capacity due to the aforementioned factors.

As used herein, the term “ototoxic” or “ototoxicity” includes, but is not limited to, any detrimental or pathologic change in the structure or function of the ear, including changes in hearing and balance. Auditory functional changes can include, but are not limited to, hearing loss or other changes in auditory threshold for any stimulus, perception of sound including recruitment (abnormal growth in the perception of loudness), ability to identify, localize, recognize, distinguish between, or process sounds, and/or distortion of sounds or any abnormality as identified by conventional auditory tests. This term also includes tinnitus (ringing or noises in the ear), which includes any perception of sound other than in response to an external signal. Further, ototoxicity includes any perceived or measured functional change in the balance or vestibular system, including, but not limited to, either induced or spontaneous vertigo, dysequilibrium, increased susceptibility to motion sickness, nausea, vomiting, nystagmus, syncope, lightheadedness, dizziness, difficulty in visual tracking secondary to vestibular or balance disorder, or abnormality as measured on any test of vestibular or balance function. Structural changes can include any intra- or extra-cellular, multicellular, or organ change in the auditory or vestibular pathways from the external ear up through and including the cortex and all pathways in between.

The term “otoprotective agent” refers to an agent that reduces, prevents, treats NIHL, CIHL, or age-induced hearing impairment or otherwise protects against hearing impairment.

The term “otodestructive” means that which causes hearing impairment.

The term “ototoxic chemotherapeutic drug” refers to a chemotherapeutic agent with an ototoxic, hearing-impairing side effect.

As used herein, the term “preventing” means to reduce the risk of occurrence of an abnormal biological or a medical event, such as hearing loss, in a cell, a tissue, a system, animal or human.

The term “treating” refers to: preventing a disease, disorder or condition from occurring in a cell, a tissue, a system, animal or human which may be predisposed to the disease, disorder and/or condition but has not yet been diagnosed as having it; stabilizing a disease, disorder or condition, i.e., arresting its development; and relieving one or more symptoms the disease, disorder or condition, i.e., causing regression of the disease, disorder and/or condition.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “as valence and stability permits” in reference to compounds disclosed herein refers to compounds that have in vitro or in vivo half-lives at room temperature of at least 12 hours, or at least 24 hours, and are preferably capable of being stored at 0° C. for a week without decomposing by more than about 10%.

The terms “half-life” or “half-lives” refer to the time required for half of a quantity of a substance to be converted to another chemically distinct species in vitro or in vivo.

The term “clathrate” refers to inclusion compounds in which the guest molecule is in a cage formed by the host molecule or by a lattice of host molecules.

The term “prodrug” refers to any compound that is converted to a more pharmacologically active compound under physiological conditions (i.e., in vivo). A common method for making a prodrug is to select moieties that are hydrolyzed under physiological conditions to provide the desired biologically active drug.

The term “metabolic derivative” refers to a compound derived by one or more in vitro or in vivo enzymatic transformations on the parent compound, wherein the resulting derivative has an ED₅₀ value as an otoprotective agent that is less than 1000×ED₅₀ value of the parent compound.

The term “ED₅₀” means the dose of a drug that produces 50% of its maximum response or effect.

The term “aminoglycoside antibiotics” includes a broad class of amino sugar-containing antibiotics well known in the art. Among the aminoglycoside agents described in the literature which are useful in the methods of the present invention are, for example, amikacin (BB-K8), butirosin, geneticin, gentamicin, kanamycin, lividomycin, neomycin, paromomycin, hybrimycin, propikacin (UK 31214), ribostamycin, seldomycin, trehalosamine, alpha.-D-mannosyl-.alpha.-D-glucosaminide, apramycin, bluensomycin, netromycin, streptomycin, tobramycin, sisomicin, destomycin, antibiotic A-396-I, dibekacin, kasugamycin, fortimicin, or derivatives, analogs or variants thereof.

The term “platinum-containing antineoplastic agents” includes a broad class of water-soluble, platinum coordination compounds well known in the art, typically having anti-tumor activity. Among the platinum-containing antineoplastic agents described in the literature which are useful in the methods of the present invention are, for example, cis-diaminedichloro-platinum(II) (cisplatin), trans-diaminedichloro-platinum(II), cis-diamine-diaquaplatinum(II)-ion, cis-diaminedichloroplatinum(II)-ion, chloro(diethylenetriamine)-platinum(II) chloride, dichloro(ethylenediamine)-platinum(II), diamine(1,1-cyclobutanedicarboxylato)-platinum(II) (carboplatin), spiroplatin, dichlorotrans-dihydroxybisisopropolamine platinum IV (iproplatin), diamine(2-ethylmalonato)platinum(II), ethylenediamine-malonatoplatinum(II), aqua(1,2-diaminodiclohexane)-sulfatoplatinum(II), (1,2-diaminocyclohexane)malonato-platinum(II), (4-carboxyphthalato)(1,2-diaminocyclo-hexane)-platinum(II), (1,2-diaminocyclohexane)-(isocitrato)platinum(II), (1,2-diaminocyclohexane)-cis(pyruvato)platinum(II), and (1,2-diaminocyclohexane)-oxalatoplatinum(II).

The term “acylamino” is art-recognized and refers to a moiety that can be represented by the general formula: 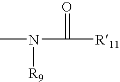
wherein R₉ is as defined above, and R′₁₁ represents a hydrogen, an alkyl, an alkenyl or —(CH₂)ₘ—R₈, where m and R₈ are as defined above.

Herein, the term “aliphatic group” refers to a straight-chain, branched-chain, or cyclic aliphatic hydrocarbon group and includes saturated and unsaturated aliphatic groups, such as an alkyl group, an alkenyl group, and an alkynyl group.

Herein, the term “ammonia equivalent” refers to a reagent employed to introduce an nitrogen moiety into a compound, which after subsequent acidic, basic, oxidative, reductive, substitution or elimination reaction or sequence of reactions is converted to an amino group.

The terms “alkenyl” and “alkynyl” refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₈, where m and R₈ are described above.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

The term “alkylthio” refers to an alkyl group, as defined above, having a sulfur radical attached thereto. In preferred embodiments, the “alkylthio” moiety is represented by one of —(S)-alkyl, —(S)-alkenyl, —(S)-alkynyl, and —(S)—(CH₂)ₘ—R₈, wherein m and R₈ are defined above. Representative alkylthio groups include methylthio, ethylthio, and the like.

The terms “amine” and “amino” are art-recognized and refer to both unsubstituted and substituted amines, e.g., a moiety that can be represented by the general formulae: 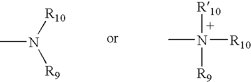
wherein R₉, R₁₀ and R′₁₀ each independently represent a hydrogen, an alkyl, an alkenyl, —(CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocyclyl or a polycyclyl; and m is zero or an integer in the range of 1 to 8. In preferred embodiments, only one of R₉ or R₁₀ can be a carbonyl, e.g., R₉, R₁₀ and the nitrogen together do not form an imide. In even more preferred embodiments, R₉ and R₁₀ (and optionally R′₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group. In certain embodiments, an amino group or an alkyl amine is basic, meaning it has a pKₐ≧7.00. The protonated forms of these functional groups have pKₐs relative to water above 7.00.

The term “amido” is art-recognized as an amino-substituted carbonyl and includes a moiety that can be represented by the general formula: 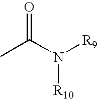
wherein R₉, R₁₀ are as defined above. Preferred embodiments of the amide will not include imides which may be unstable.

The term “aralkyl”, as used herein, refers to an alkyl group substituted with an aryl group (e.g., an aromatic or heteroaromatic group).

The term “aryl” as used herein includes 5-, 6-, and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphate, phosphonate, phosphinate, carbonyl, carboxyl, silyl, sulfamoyl, sulfinyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The term “carbocycle”, as used herein, refers to an aromatic or non-aromatic ring in which each atom of the ring is carbon. The carbocycle can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, or the like.

The term “carbonyl” is art-recognized and includes such moieties as can be represented by the general formula: 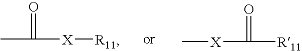
wherein X is a bond or represents an oxygen or a sulfur, and R₁₁ represents a hydrogen, an alkyl, an alkenyl, —(CH₂)ₘ—R₈ or a pharmaceutically acceptable salt, R′₁₁ represents a hydrogen, an alkyl, an alkenyl or —(CH₂)ₘ—R₈, where m and R₈ are as defined above. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R₁₁′ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The term “heteroatom” as used herein means an atom of any element other than carbon or hydrogen. Preferred heteroatoms are boron, nitrogen, oxygen, phosphorus, sulfur and selenium.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, or the like.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

A “phosphonamidite” can be represented in the general formula: 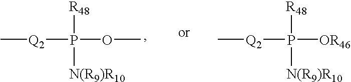
wherein R₉ and R₁₀ are as defined above, Q₂ represents O, S or N, and R₄₈ represents a lower alkyl or an aryl, Q₂ represents O, S or N.

A “phosphoramidite” can be represented in the general formula: 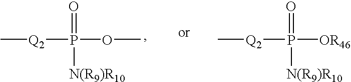
wherein R₉ and R₁₀ are as defined above, and Q₂ represents O, S or N.

A “phosphoryl” can in general be represented by the formula: 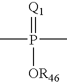
wherein Q₁ represented S or O, and R₄₆ represents hydrogen, a lower alkyl or an aryl. When used to substitute, for example, an alkyl, the phosphoryl group of the phosphorylalkyl can be represented by the general formula: 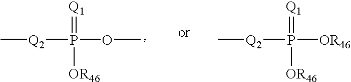
wherein Q₁ represented S or O, and each R₄₆ independently represents hydrogen, a lower alkyl or an aryl, Q₂ represents O, S or N. When Q₁ is an S, the phosphoryl moiety is a “phosphorothioate”.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, or the like.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

A “selenoalkyl” refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R₈, m and R₈ being defined above.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

The term “sulfamoyl” is art-recognized and includes a moiety that can be represented by the general formula: 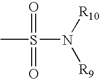
in which R₉ and R₁₀ are as defined above.

The term “sulfate” is art recognized and includes a moiety that can be represented by the general formula: 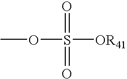
in which R₄₁ is as defined above.

The term “sulfonamido” is art recognized and includes a moiety that can be represented by the general formula: 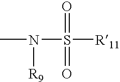
in which R₉ and R₁₁ are as defined above.

The term “sulfonate” is art-recognized and includes a moiety that can be represented by the general formula: 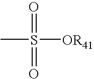
in which R₄₁ is an electron pair, hydrogen, alkyl, cycloalkyl, or aryl.

The terms “sulfoxido” or “sulfinyl”, as used herein, refers to a moiety that can be represented by the general formula: 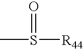
in which R₄₄ is selected from the group consisting of hydrogen, alkyl, alkenyl, alkynyl, cycloalkyl, heterocyclyl, aralkyl, or aryl.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g., alkyl, m, n, etc., when it occurs more than once in any structure, is intended to be independent of its definition elsewhere in the same structure.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

Certain compounds of the present invention may exist in particular geometric or stereoisomeric forms. The present invention contemplates all such compounds, including cis- and trans-isomers, (R)- and (S)-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts may be formed with an appropriate optically active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof, wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

C. Exemplary Embodiments

Otoprotective compounds would be useful in the context of hazards posed by loud noises in certain occupational or recreational activities or injuries arising from ototoxic chemicals including counteracting ototoxic side-effects associated with certain chemotherapeutic regimes, or improving quality of life in aging populations experiencing progressive hearing impairment. The present invention contemplates uses of such otoprotective compounds both for hearing loss and hearing impairment.

Accordingly, in one embodiment, the present invention describes a method for preventing, reducing, or otherwise treating hearing impairment due to NIHL, aging, or CIHL comprising administering to a patient a thiourea or a pharmaceutically acceptable salt, tautomer, solvate, clathrate, prodrug or metabolic derivative thereof, having a structure according to Formula I: 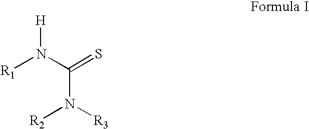
wherein, as valence and stability permit,
R₁, R₂ and R₃, independently for each occurrence, represent hydrogen, alkyl, alkenyl, alkynyl, alkylthio, imine, amide, cyano, isocyano, carbonyl, carboxyl, carboxamide, alkylsulfonyl, arylsulfonyl, ketone, aldehyde, ester, heteroalkyl, nitrile, amidine, acetal, ketal, substituted or unsubstituted aryl, substituted or unsubstituted heteroaryl, aziridine, carbamate, imide, urea, thiourea, or R₁ and R₂ taken together form a substituted or unsubstituted aryl, heteroaryl, carbocyclyl, or heterocyclyl ring having 4 to 8 members, or R₂ and R₃ taken together form a substituted or unsubstituted aryl, heteroaryl, carbocyclyl, or heterocyclyl ring having 4 to 8 members.

In one embodiment of this composition, R₁ is connected to R₂ or R₃ by a covalent, ionic or metal coordination bond to form a substituted or unsubstituted aryl, cycloalkyl, cycloalkenyl, heterocyclyl, polycyclyl or cyclic metal complex.

In another embodiment of this composition, R₂ and R₃ are connected by a covalent, ionic or metal coordination bond to form a substituted or unsubstituted aryl, cycloalkyl, cycloalkenyl, heterocyclyl, polycyclyl or cyclic metal complex.

In another embodiment, the present invention describes a method for preventing, reducing, or otherwise treating hearing impairment due to NIHL, aging, or CIHL comprising administering to a patient a compound having a structure depicted in Formula II, or a pharmaceutically acceptable salt, tautomer, solvate, or clathrate thereof: 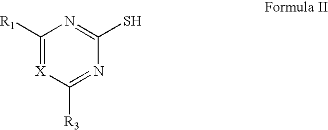
wherein, as valence and stability permit,
X represents C—R₂;
R₁, R₂ and R₃, independently for each occurrence, represent hydrogen, alkyl, alkenyl, alkynyl, alkylthio, imine, amide, cyano, isocyano, carbonyl, carboxyl, carboxamide, alkylsulfonyl, arylsulfonyl, ketone, aldehyde, ester, heteroalkyl, nitrile, amidine, acetal, ketal, substituted or unsubstituted aryl, substituted or unsubstituted heteroaryl, aziridine, carbamate, imide, urea, thiourea, or R₁ and R₂ taken together form a substituted or unsubstituted aryl, heteroaryl, carbocyclyl, or heterocyclyl ring having 4 to 8 members, or R₂ and R₃ taken together form a substituted or unsubstituted aryl, heteroaryl, carbocyclyl, or heterocyclyl ring having 4 to 8 members.

In one embodiment of this composition, at least one of R₁, R₂, and R₃ represents a sulfhydryl or alkylthio group.

In one embodiment of this composition, X represents CH, R₁ represents H and R₃ represents a propyl group.

In another embodiment, X represents a C—R₂; and R₃ represents a hydroxyl.

In certain embodiments of Formulae I and II, at least one of R₁ and R₂ represents a hydrogen.

In certain embodiments of Formulae I and II, R₁ and R₂ represent lower straight-chained or branched alkyls containing up to 6 carbons such as methyl, ethyl, n-propyl, isopropyl, n-butyl, sec-butyl, n-pentyl and n-hexyl moieties.

In certain embodiments of Formulae I and II, R₂ represents a carboxyl or a pharmaceutically acceptable derivative thereof.

One aspect of the invention is a method for preventing, reducing or treating ototoxicity in a subject undergoing treatment with an ototoxic chemotherapeutic drug selected from an aminoglycoside antibiotic, a platinum-containing antineoplastic agent, certain quinine-like compounds or an ototoxic diuretic drug, by administering to the subject in need of such treatment a therapeutic dosage of an otoprotective agent disclosed herein.

Another aspect of the present invention relates to methods for augmenting treatments which require administration of an ototoxic chemical or chemotherapeutic agent, comprising administering an effective amount of an otoprotective agent to prevent, reduce, or treat the hearing impairment caused by the ototoxic agent. In certain embodiments, the otoprotective agent and chemotherapeutic agent may be provided in a single pharmaceutical preparation, e.g., as a single dosage form. In other embodiments, the two agents can be provided as a kit in which each is provided in separate dosages, along with instructions for co-administering the two agents.

In one embodiment, the composition may be administered prior to, simultaneously with, or subsequent to administration of said ototoxic chemotherapeutic agent.

In certain embodiments, the invention provides a method wherein a therapeutically effective amount of otoprotective agent is administered with each dose of ototoxic chemotherapeutic agent, or at specified intervals throughout the treatment course, or at the beginning of the treatment course. In certain embodiments, the invention provides a method wherein a therapeutically effective amount of otoprotective composition is administered between 72 hours before and 36 hours after treatment with said ototoxic chemotherapeutic drug.

In certain embodiments, the invention provides a method wherein a therapeutically effective amount of otoprotective composition is administered to prevent, reduce, or otherwise treat hearing impairment due to NIHL, wherein the otoprotective agent is administered between 72 hours before and 36 hours after otodestructive noise.

Representative aminoglycoside antibiotics include, but are not limited to, amikacin (BB-K8), butirosin, geneticin, gentamicin, kanamycin, lividomycin, neomycin, paromomycin, hybrimycin, propikacin (UK 31214), ribostamycin, seldomycin, trehalosamine, α-D-mannosyl-α-D-glucosaminide, apramycin, bluensomycin, netromycin, streptomycin, tobramycin, sisomicin, destomycin, Antibiotic A-396-I, dibekacin, kasugamycin, fortimicin, or derivatives, analogs or variants thereof.

Representative platinum-containing antineoplastic agents include, but are not limited to, cis-diaminedichloroplatinum(II) (cisplatin), trans-diaminedichloroplatinum(II), cis-diamine-diaquaplatinum(II)-ion, chloro(diethylenetriamine)-platinum(II) chloride, dichloro(ethylene-diamine)-platinum(II), diamine(1,1-cyclobutanedi-carboxylato)-platinum(II), spiroplatin, dichlorotrans-dihydroxybisisopropolamine platinum IV (iproplatin), diamine(2-ethylmalonato)-platinum(II), ethylenediamine-malonatoplatinum(II), aqua(1,2-diaminodyclohexane)-sulfatoplatinum(II), (1,2-diaminocyclohexane)malonato-platinum(II), (4-carboxyphthalato)(1,2-diaminocyclo-hexane)-platinum(II), (1,2-diaminocyclohexane)-(isocitrato)platinum(II), (1,2-diaminocyclohexane)-cis(pyruvato)platinum(II), or (1,2-diaminocyclohexane)-oxalatoplatinum(II).

Another aspect of the invention is a pharmaceutical dosage form comprising a therapeutically effective amount of an otoprotective compound, or a pharmaceutically acceptable salt, tautomer solvate, clathrate, prodrug or metabolic derivative thereof. In one embodiment, the dosage form is a tablet, capsule or an oral solution. In another embodiment, the dosage may be adapted for intravenous infusion, parenteral delivery or oral delivery.

In another embodiment, the therapeutically effective amount of the compound is in the range of from about 0.1 mg/kg body weight to about 500 mg/kg body weight, from about 1 mg/kg body weight to about 400 mg/kg body weight, from about 10 mg/kg body weight to about 100 mg/kg body weight, or even from about 10 mg/kg body weight to about 75 mg/kg body weight.

Another aspect of the present invention is a method for conducting a pharmaceutical business, comprising:
a. manufacturing a preparation of any of the otoprotective compositions disclosed herein, or a kit comprising an otoprotective agent and an ototoxic chemotherapeutic drug; and
b. marketing to healthcare providers the benefits of using the preparation or kit in the treatment of ototoxic side-effects associated with said ototoxic chemotherapeutic drugs.

In certain embodiments, the invention provides a method for conducting a pharmaceutical business, comprising:
a. providing a distribution network for selling said preparation or kit; and
b. providing instruction material to patients or physicians for using the preparation or kit to treat ototoxic side-effects associated with said chemotherapeutic drugs.

In certain embodiments, the invention also provides a method for conducting a pharmaceutical business, comprising:
a. determining an appropriate formulation and dosage of an otoprotective agent and an ototoxic chemotherapeutic agent such as ototoxic aminoglycoside antibiotic, platinum-containing antineoplastic agent, a pharmaceutical composition comprising ototoxic quinine-like compounds or an ototoxic diuretic drug to be co-administered in the treatment of a bacterial infection, cancer chemotherapy or a disorder requiring ototoxic diuretic or an ototoxic quinine-type compound;
b. conducting therapeutic profiling of formulations identified in step (a), for efficacy and toxicity in animals; and
c. providing a distribution network for selling a preparation identified in step (b) as having an acceptable therapeutic profile.

In still further embodiments, the method includes an additional step of providing a sales group for marketing the preparation to healthcare providers.

In yet other embodiments, the invention provides a method for conducting a pharmaceutical business, comprising:
a. determining an appropriate formulation and dosage of an otoprotective agent and an ototoxic chemotherapeutic agent such as ototoxic aminoglycoside antibiotic, platinum-containing antineoplastic agent, a pharmaceutical composition comprising ototoxic quinine-like compounds or an ototoxic diuretic drug to be co-administered in the treatment of a bacterial infection, cancer chemotherapy or a disorder requiring ototoxic diuretic or an ototoxic quinine-type compound; and
b. licensing, to a third party, the rights for further development and sale of the formulation.
   D. Exemplary Formulations

In another aspect, the present invention provides pharmaceutical compositions. The composition for use in the subject method may be conveniently formulated for administration with a biologically acceptable medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the otoprotection, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”.

Pharmaceutical formulations of the present invention can also include veterinary compositions, e.g., pharmaceutical preparations of the otoprotective agent suitable for veterinary uses, e.g., for the treatment of livestock or domestic animals, e.g., dogs.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a drug at a particular target site.

The preparations of the present invention may be given orally, parenterally, topically, or rectally. They are, of course, given by forms suitable for each administration route. For example, they are administered in tablets or capsule form, by injection, inhalation, eye lotion, ointment, suppository, controlled release patch, etc.; administration by injection, infusion or inhalation; topical by lotion or ointment; and rectal by suppositories. Oral and topical administrations are preferred.

The phrases “parenteral administration” or “administered parenterally” as used herein mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular composition employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect, and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous and subcutaneous doses of the compounds of this invention for a patient will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable and/or sterile carriers and can also be administered in conjunction with other antimicrobial agents such as penicillins, cephalosporins, aminoglycosides and glycopeptides. Conjoint therapy includes sequential, simultaneous, and separate administration of the active compound in a way that the therapeutical effects of the first administered one is not entirely dissipated when the subsequent is administered.

The phrase “therapeutically effective amount” as used herein means that amount of a compound, material, or composition comprising a compound of the present invention which is effective for producing some otoprotection, at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its analogs, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, the pharmaceutical preparation is non-pyrogenic, i.e., does not substantially elevate the body temperature of a patient.

As set out above, certain embodiments of the present composition may contain a basic functional group, such as amino or alkylamino, and are, thus, capable of forming pharmaceutically acceptable salts with pharmaceutically acceptable acids. The term “pharmaceutically acceptable salts” in this respect refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Pharmacological dosages or formulations of the present invention include those suitable for oral, nasal, topical (including buccal and sublingual), rectal, vaginal and/or parenteral administration. The dosages may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active ingredient.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose analogs, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the composition in the proper medium. Absorption enhancers can also be used to increase the flux of the composition across the skin. The rate of such flux can be controlled by either providing a rate-controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient, or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” 0 and B books, Corvallis, Ore., U.S.A., 1977).

### E. EXAMPLES

### Example 1

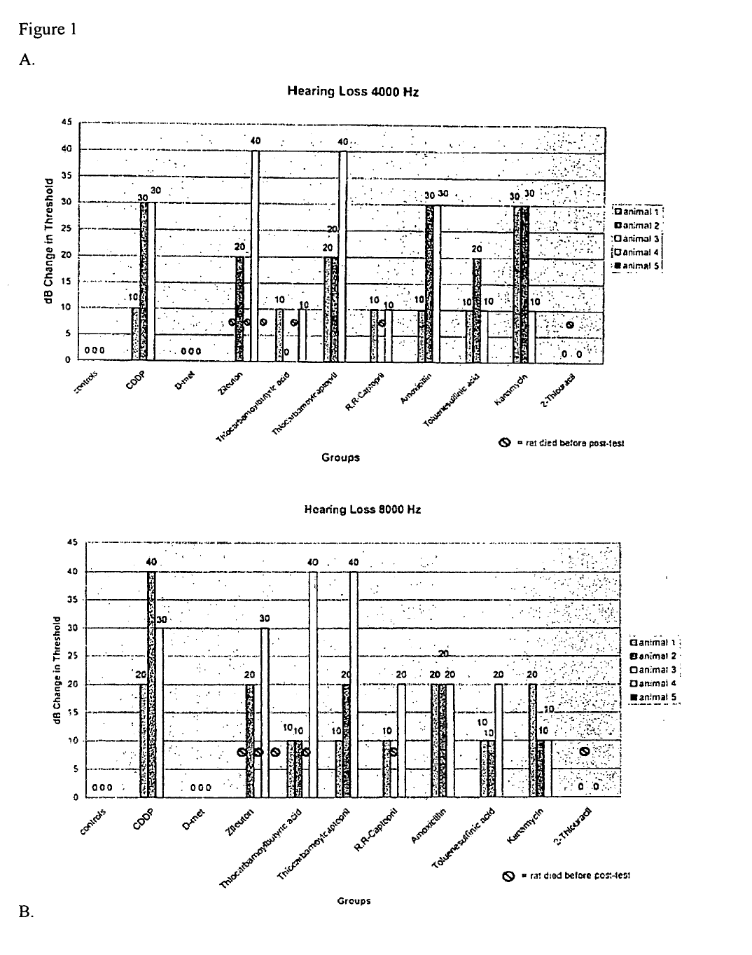

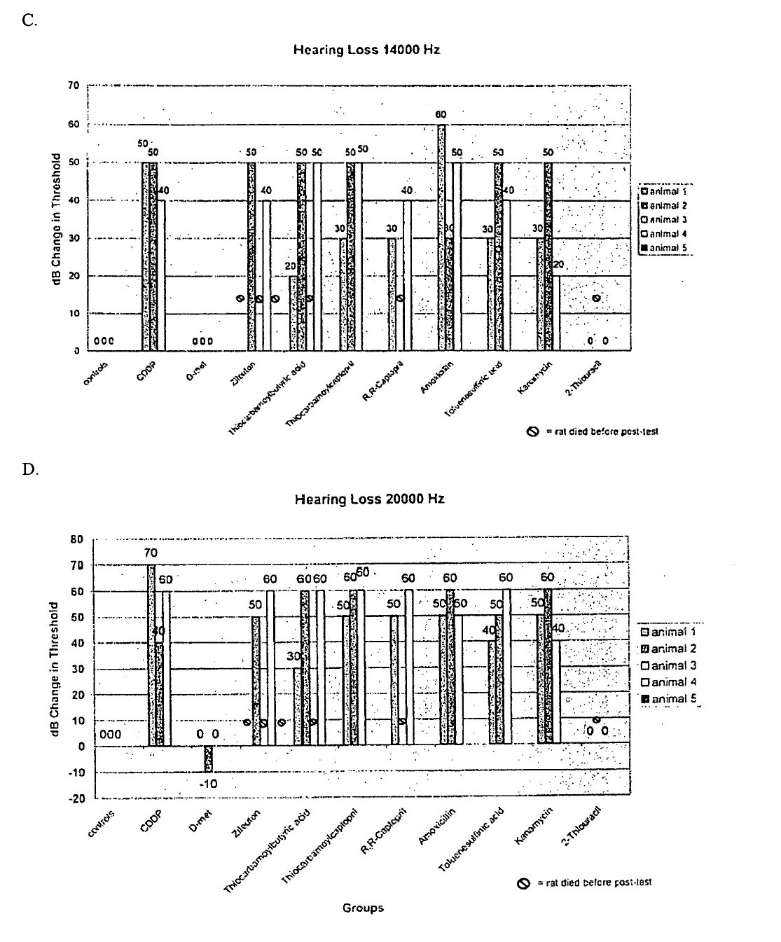

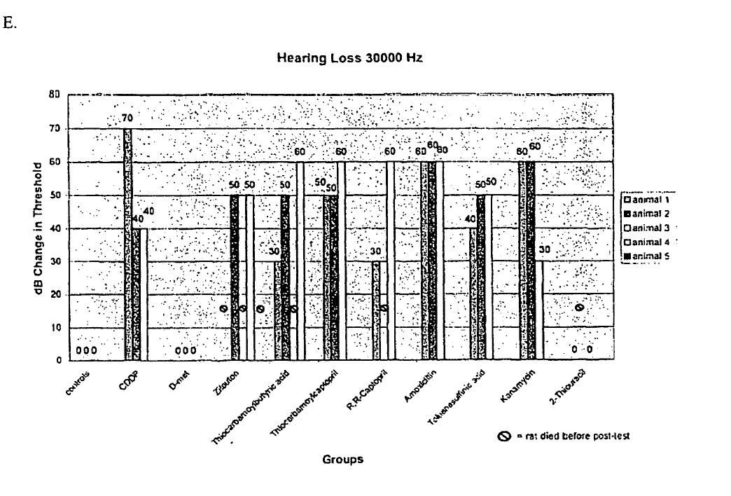

Screening experiments were carried out according to Auditory Brainstem testing (ABR) protocols described in U.S. Pat. No. 6,187,817. Five groups, each consisting of 5 Wistar rats, anesthetized, and were injected with the test compounds, dissolved in normal saline, pH 6.6, to give a final concentration of 300 mg/kg. The groups of animals, except for one untreated control group, received cisplatin by slow i.p. infusion (dissolved in normal saline, pH 6.6) to give a final concentration of 16 mg/kg. The first ABR testing was carried out just prior to administration of cisplatin or saline, but after the administration of the test compounds. The rats were again tested three days later and the change in threshold sensitivity (dB) over this period was assessed at a range of stimulator frequencies. See FIG. 1.

### Example 2

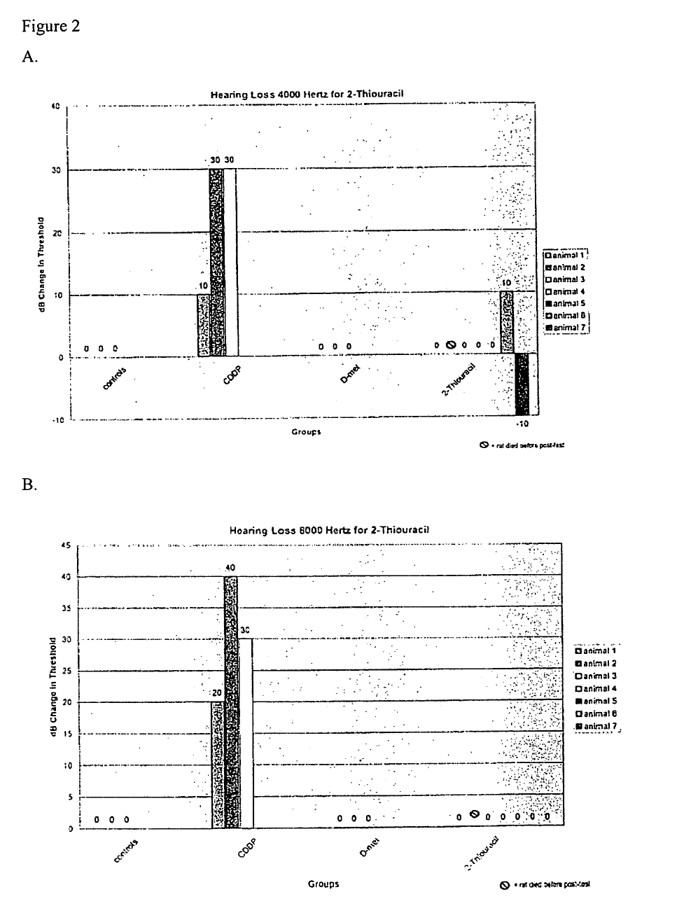

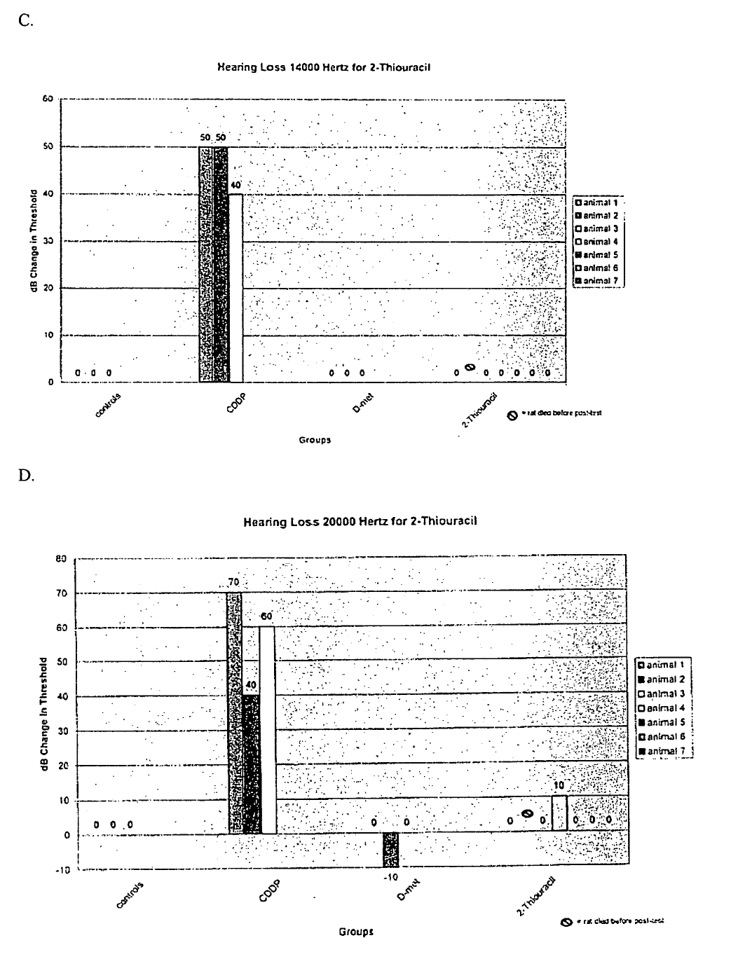

The otoprotective efficacies of D-methionine and 2-thiouracil were compared. The experiments were done as described above except that each group contained seven rats. The control animal was treated with only saline and received neither protective agent nor cisplatin (CDDP). The CDDP group was treated with cisplatin (16 mg/kg) and saline, cisplatin (16 mg/kg) and D-methionine (300 mg/kg), or cisplatin (16 mg/kg) and 2-thiouracil (300 mg/kg). ABR testing was done prior to cisplatin treatment to establish a baseline and the again three days post cisplatin treatment. See FIG. 2.

### Example 3

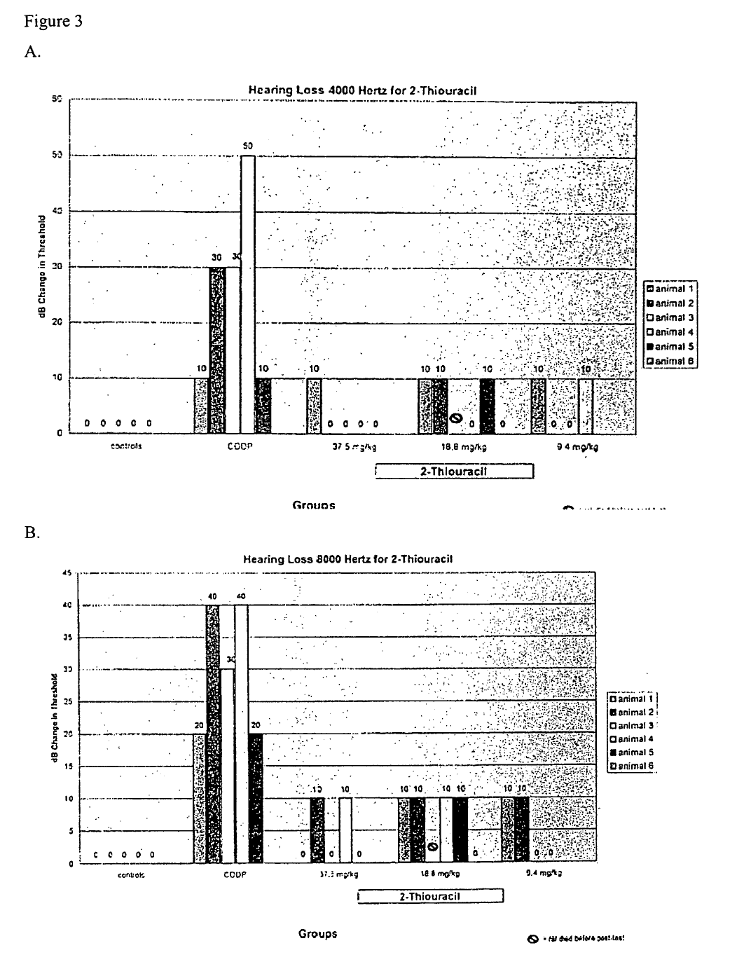

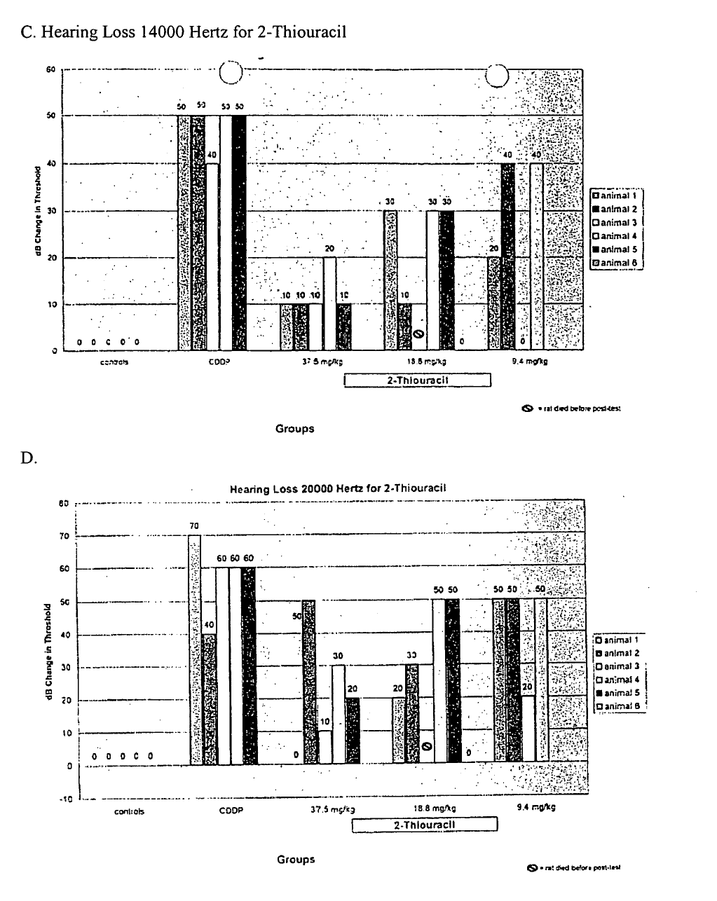

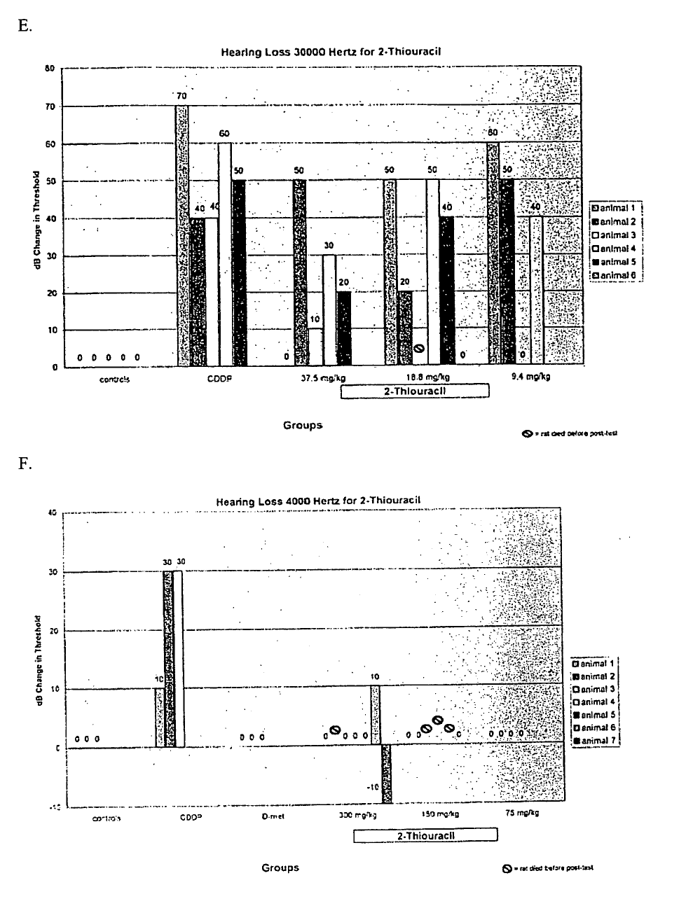

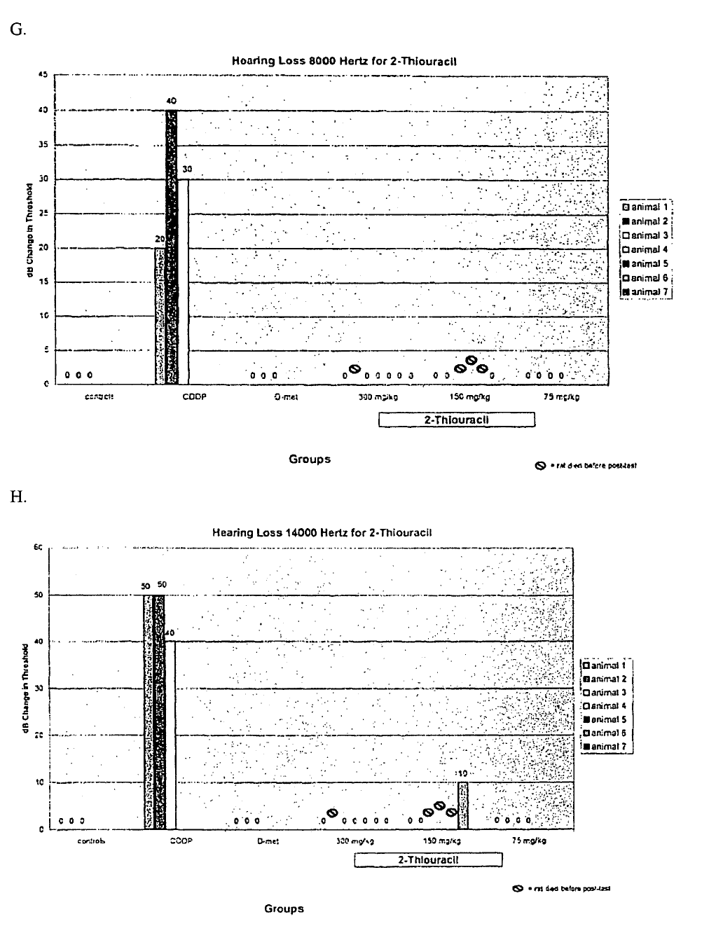

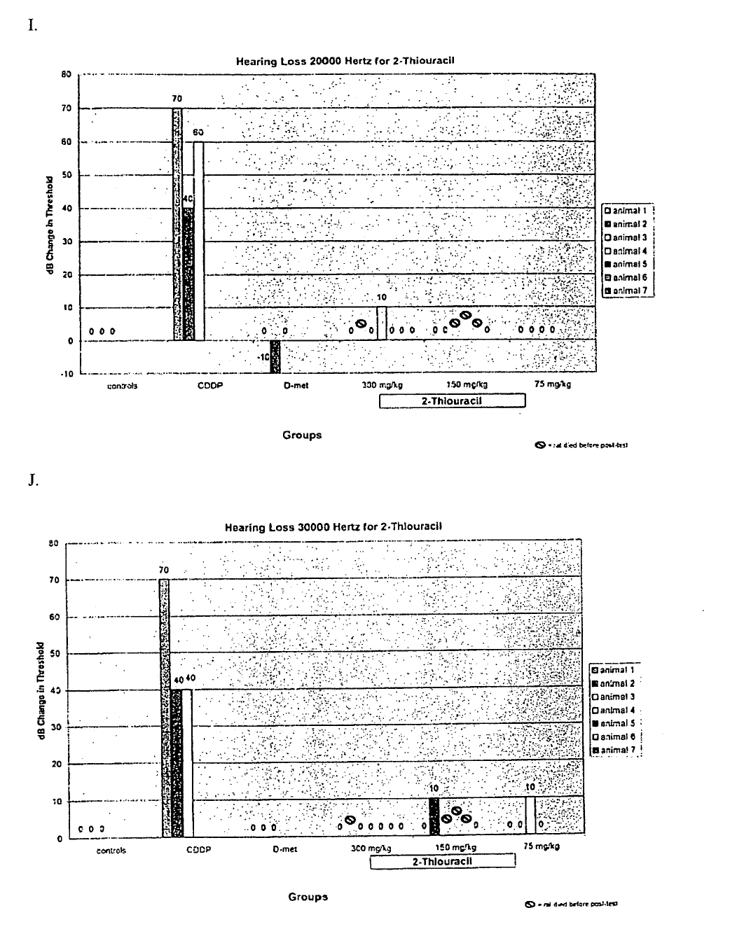

Experiments were conducted to determine the minimal amount of 2-thiouracil needed to prevent cisplatin-induced hearing loss. The experiments were performed as described in Example 1, except that varying levels of 2-thiouracil were used. See FIG. 3.

1. A method for preventing, reducing, or otherwise treating hearing impairment due to noise-induced hearing loss (NIHL), aging, or chemical-induced hearing loss (CIHL), comprising administering to a subject a compound, or a pharmaceutically acceptable salt, tautomer, solvate, clathrate, prodrug or metabolic derivative thereof, having a structure according to Formula I: 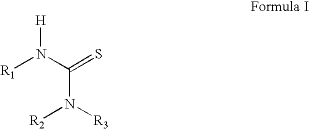
wherein, as valence and stability permit,
R₁, R₂ and R₃, independently for each occurrence, represent hydrogen, alkyl, alkenyl, alkynyl, alkylthio, imine, amide, cyano, isocyano, carbonyl, carboxyl, carboxamide, alkylsulfonyl, arylsulfonyl, ketone, aldehyde, ester, heteroalkyl, nitrile, amidine, acetal, ketal, substituted or unsubstituted aryl, substituted or unsubstituted heteroaryl, aziridine, carbamate, imide, urea, or thiourea, or R₁ and R₂ taken together form a substituted or unsubstituted aryl, heteroaryl, carbocyclyl, or heterocyclyl ring having 4 to 8 members, or R₂ and R₃ taken together form a substituted or unsubstituted aryl, heteroaryl, carbocyclyl, or heterocyclyl ring having 4 to 8 members.

2. The method of claim 1, wherein R₁ and R₂, taken together form a substituted or unsubstituted aryl, a cycloalkyl, cycloalkenyl, heterocyclyl, polycyclyl, or cyclic metal complex.

3. The method of claim 1, wherein R₂ and R₃, taken together form a substituted or unsubstituted aryl, cycloalkyl, cycloalkenyl, heterocyclyl, polycyclyl, or cyclic metal complex.

4. A method for preventing, reducing, or otherwise treating hearing impairment due to NIHL, aging, or CIHL, comprising administering to a subject a compound having a structure of Formula II, or a pharmaceutically acceptable salt, tautomer, solvate, or clathrate thereof: 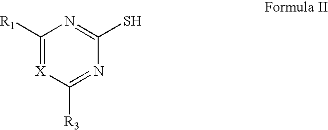
wherein, as valence and stability permit,
X represents C—R₂;
R₁, R₂ and R₃, independently for each occurrence, represent hydrogen, alkyl, alkenyl, alkynyl, alkylthio, imine, amide, cyano, isocyano, carbonyl, carboxyl, carboxamide, alkylsulfonyl, arylsulfonyl, ketone, aldehyde, ester, heteroalkyl, nitrile, amidine, acetal, ketal, substituted or unsubstituted aryl, substituted or unsubstituted heteroaryl, aziridine, carbamate, imide, urea, or thiourea, or R₁ and R₂ taken together form a substituted or unsubstituted aryl, heteroaryl, carbocyclyl, or heterocyclyl ring having 4 to 8 members, or R₂ and R₃ taken together form a substituted or unsubstituted aryl, heteroaryl, carbocyclyl, or heterocyclyl ring having 4 to 8 members.

5. The method of claim 4, wherein at least one of R₁, R₂, and R₃ represents a sulfhydryl or alkylthio group.

6. The method of claim 4, wherein
X represents a C—R₂; and
R₃ represents a hydroxyl.

7. The method of claim 4, wherein X represents C—R₂ and at least one of R₁ or R₂ represents hydrogen.

8. The method of claim 4, wherein X represents C—R₂ and R₁ and R₂ represent lower straight-chained or branched C, to C₆ alkyls.

9. The method of claim 4, wherein X represents CH, R₁ represents H, and R₃ represents a propyl group.

10. The method of claim 4, wherein X represents C—R₂ and R₂ represents a carboxyl or a pharmaceutically acceptable derivative thereof.

11. A method for preventing or treating hearing impairment in a subject undergoing treatment with an ototoxic chemotherapeutic drug selected from an aminoglycoside antibiotic, a platinum-containing antineoplastic agent, certain quinine-like compounds or an ototoxic diuretic drug, comprising administering to the subject in need of such treatment a therapeutically effective amount of a compound represented by formula I: 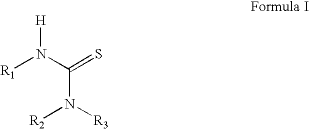
wherein, as valence and stability permit,
R₁, R₂ and R₃, independently for each occurrence, represent hydrogen, alkyl, alkenyl, alkynyl, alkylthio, imine, amide, cyano, isocyano, carbonyl, carboxyl, carboxamide, alkylsulfonyl, arylsulfonyl, ketone, aldehyde, ester, heteroalkyl, nitrile, amidine, acetal, ketal, substituted or unsubstituted aryl, substituted or unsubstituted heteroaryl, aziridine, carbamate, imide, urea, or thiourea, or R₁ and R₂ taken together form a substituted or unsubstituted aryl, heteroaryl, carbocyclyl, or heterocyclyl ring having 4 to 8 members, or R₂ and R₃ taken together form a substituted or unsubstituted aryl, heteroaryl, carbocyclyl, or heterocyclyl ring having 4 to 8 members; without reducing the chemotherapeutic drug's efficacy.

12. A method for preventing or treating hearing impairment in a subject undergoing treatment with an ototoxic chemotherapeutic drug selected from an aminoglycoside antibiotic, a platinum-containing antineoplastic agent, certain quinine-like compounds or an ototoxic diuretic drug, comprising administering to the subject in need of such treatment a therapeutically effective amount of a compound represented by formula II: 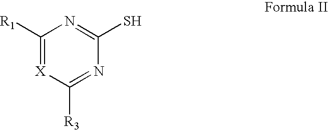
wherein, as valence and stability permit,
X represents C—R₂;
R₁, R₂ and R₃, independently for each occurrence, represent hydrogen, alkyl, alkenyl, alkynyl, alkylthio, imine, amide, cyano, isocyano, carbonyl, carboxyl, carboxamide, alkylsulfonyl, arylsulfonyl, ketone, aldehyde, ester, heteroalkyl, nitrile, amidine, acetal, ketal, substituted or unsubstituted aryl, substituted or unsubstituted heteroaryl, aziridine, carbamate, imide, urea, or thiourea, or R₁ and R₂ taken together form a substituted or unsubstituted aryl, heteroaryl, carbocyclyl, or heterocyclyl ring having 4 to 8 members, or R₂ and R₃ taken together form a substituted or unsubstituted aryl, heteroaryl, carbocyclyl, or heterocyclyl ring having 4 to 8 members; without reducing the chemotherapeutic drug's efficacy.

13. The method of any of claims 11-12, wherein said compound is administered prior to, simultaneously with, or subsequent to administration of said ototoxic chemotherapeutic drug.

14. The method of any of claims 11-12, wherein a therapeutically effective amount of the compound is administered with each dose of ototoxic chemotherapeutic agent, at specified intervals throughout the treatment course, or at the beginning of the treatment course.

15. The method of any of claims 11-12, wherein a the compound is administered between 72 hours before and 36 hours after treatment with said ototoxic chemotherapeutic drug.

16. The method of any of claims 11-12, wherein the drug is an aminoglycoside antibiotic selected from amikacin (BB-K8), butirosin, geneticin, gentamicin, kanamycin, lividomycin, neomycin, paromomycin, hybrimycin, propikacin (UK 31214), ribostamycin, seldomycin, trehalosamine, α-D-mannosyl-α-D-glucosaminide, apramycin, bluensomycin, netromycin, streptomycin, tobramycin, sisomicin, destomycin, Antibiotic A-396-I, dibekacin, kasugamycin, fortimicin, or derivative or analog or variants thereof.

17. The method of any of claims 11-12, wherein the drug is a platinum-containing antineoplastic agent selected from cis-diaminedichloroplatinum(II) (cisplatin), trans-diaminedichloroplatinum(II), cis-diamine-diaquaplatinum(II)-ion, chloro(diethylenetriamine)-platinum(II) chloride, dichloro(ethylene-diamine)-platinum(II), diamine(1,1-cyclobutanedi-carboxylato)-platinum(II), spiroplatin, dichlorotrans-dihydroxybisisopropolamine platinum IV (iproplatin), diamine(2-ethylmalonato)-platinum(II), ethylenediamine-malonatoplatinum(II), aqua(1,2-diaminodyclohexane)-sulfatoplatinum(II), (1,2-diaminocyclohexane)malonato-platinum(II), (4-carboxyphthalato)(1,2-diaminocyclo-hexane)-platinum(II), (1,2-diaminocyclohexane)-(isocitrato)platinum(II), (1,2-diaminocyclohexane)-cis(pyruvato)platinum(II), or (1,2-diaminocyclohexane)-oxalatoplatinum(II).

18. A method to prevent, reduce, or otherwise treat hearing impairment due to NIHL, comprising administering a therapeutically effective amount of a compound represented by formula I: 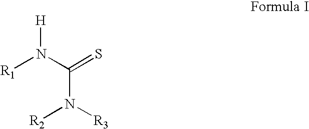
wherein, as valence and stability permit,
R₁, R₂ and R₃, independently for each occurrence, represent hydrogen, alkyl, alkenyl, alkynyl, alkylthio, imine, amide, cyano, isocyano, carbonyl, carboxyl, carboxamide, alkylsulfonyl, arylsulfonyl, ketone, aldehyde, ester, heteroalkyl, nitrile, amidine, acetal, ketal, substituted or unsubstituted aryl, substituted or unsubstituted heteroaryl, aziridine, carbamate, imide, urea, or thiourea, or R₁ and R₂ taken together form a substituted or unsubstituted aryl, heteroaryl, carbocyclyl, or heterocyclyl ring having 4 to 8 members, or R₂ and R₃ taken together form a substituted or unsubstituted aryl, heteroaryl, carbocyclyl, or heterocyclyl ring having 4 to 8 members.

19. A method to prevent, reduce, or otherwise treat hearing impairment due to NIHL, comprising administering a therapeutically effective amount of a compound represented by formula II: 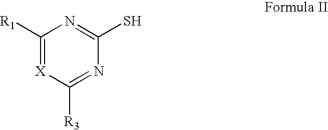
wherein, as valence and stability permit,
X represents C—R₂;
R₁, R₂ and R₃, independently for each occurrence, represent hydrogen, alkyl, alkenyl, alkynyl, alkylthio, imine, amide, cyano, isocyano, carbonyl, carboxyl, carboxamide, alkylsulfonyl, arylsulfonyl, ketone, aldehyde, ester, heteroalkyl, nitrile, amidine, acetal, ketal, substituted or unsubstituted aryl, substituted or unsubstituted heteroaryl, aziridine, carbamate, imide, urea, or thiourea, or R₁ and R₂ taken together form a substituted or unsubstituted aryl, heteroaryl, carbocyclyl, or heterocyclyl ring having 4 to 8 members, or R₂ and R₃ taken together form a substituted or unsubstituted aryl, heteroaryl, carbocyclyl, or heterocyclyl ring having 4 to 8 members.

20. The method of any of claims 18 or 19, wherein the compound is administered between 72 hours before and 36 hours after an otodestructive noise.

21. A pharmaceutical dosage form comprising a therapeutically effective amount of the compound of represented by formula I: 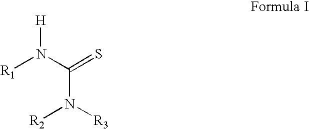
wherein, as valence and stability permit,
R₁, R₂ and R₃, independently for each occurrence, represent hydrogen, alkyl, alkenyl, alkynyl, alkylthio, imine, amide, cyano, isocyano, carbonyl, carboxyl, carboxamide, alkylsulfonyl, arylsulfonyl, ketone, aldehyde, ester, heteroalkyl, nitrile, amidine, acetal, ketal, substituted or unsubstituted aryl, substituted or unsubstituted heteroaryl, aziridine, carbamate, imide, urea, or thiourea, or R₁ and R₂ taken together form a substituted or unsubstituted aryl, heteroaryl, carbocyclyl, or heterocyclyl ring having 4 to 8 members, or R₂ and R₃ taken together form a substituted or unsubstituted aryl, heteroaryl, carbocyclyl, or heterocyclyl ring having 4 to 8 members.

22. A pharmaceutical dosage form comprising a therapeutically effective amount of the compound of represented by formula II: 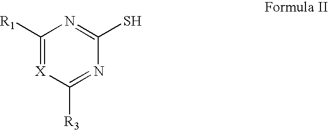
wherein, as valence and stability permit,
X represents C—R₂;
R₁, R₂ and R₃, independently for each occurrence, represent hydrogen, alkyl, alkenyl, alkynyl, alkylthio, imine, amide, cyano, isocyano, carbonyl, carboxyl, carboxamide, alkylsulfonyl, arylsulfonyl, ketone, aldehyde, ester, heteroalkyl, nitrile, amidine, acetal, ketal, substituted or unsubstituted aryl, substituted or unsubstituted heteroaryl, aziridine, carbamate, imide, urea, or thiourea, or R₁ and R₂ taken together form a substituted or unsubstituted aryl, heteroaryl, carbocyclyl, or heterocyclyl ring having 4 to 8 members, or R₂ and R₃ taken together form a substituted or unsubstituted aryl, heteroaryl, carbocyclyl, or heterocyclyl ring having 4 to 8 members.

23. The dosage form of any of claims 21 or 22, wherein said dosage form is a tablet, a capsule, or an oral solution.

24. The dosage form of any of claims 21 or 22, wherein said dosage form is adapted for intravenous infusion, parenteral delivery, or oral delivery.

25. The dosage form of any of claims 21 or 22, wherein said therapeutically effective amount of the compound is in the range of about 0.1 mg/kg body weight to about 500 mg/kg body weight.

26. The dosage form of any of claims 21 or 22, wherein said therapeutically effective amount of the compound is in the range of about 1 mg/kg body weight to about 400 mg/kg body weight.

27. The dosage form of any of claims 21 or 22, wherein said therapeutically effective amount of the compound is in the range of about 10 mg/kg body weight to about 100 mg/kg body weight.

28. The dosage form of any of claims 21 or 22, wherein said effective amount of compound is in the range of about 10 mg/kg body weight to about 75 mg/kg body weight.

29. A pharmaceutical formulation comprising a therapeutically effective amount of a compound of formula I: 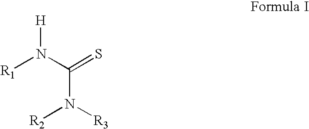
wherein, as valence and stability permit,
R₁, R₂ and R₃, independently for each occurrence, represent hydrogen, alkyl, alkenyl, alkynyl, alkylthio, imine, amide, cyano, isocyano, carbonyl, carboxyl, carboxamide, alkylsulfonyl, arylsulfonyl, ketone, aldehyde, ester, heteroalkyl, nitrile, amidine, acetal, ketal, substituted or unsubstituted aryl, substituted or unsubstituted heteroaryl, aziridine, carbamate, imide, urea, or thiourea, or R₁ and R₂ taken together form a substituted or unsubstituted aryl, heteroaryl, carbocyclyl, or heterocyclyl ring having 4 to 8 members, or R₂ and R₃ taken together form a substituted or unsubstituted aryl, heteroaryl, carbocyclyl, or heterocyclyl ring having 4 to 8 members;
formulated together with an ototoxic chemotherapeutic agent, either as a preparation or a kit, without diminishing the efficacy of the chemotherapeutic agent.

30. A pharmaceutical formulation, wherein a therapeutically effective amount of a compound represented by formula II: 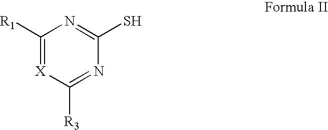
wherein, as valence and stability permit,
X represents C—R₂;
R₁, R₂ and R₃, independently for each occurrence, represent hydrogen, alkyl, alkenyl, alkynyl, alkylthio, imine, amide, cyano, isocyano, carbonyl, carboxyl, carboxamide, alkylsulfonyl, arylsulfonyl, ketone, aldehyde, ester, heteroalkyl, nitrile, amidine, acetal, ketal, substituted or unsubstituted aryl, substituted or unsubstituted heteroaryl, aziridine, carbamate, imide, urea, or thiourea, or R₁ and R₂ taken together form a substituted or unsubstituted aryl, heteroaryl, carbocyclyl, or heterocyclyl ring having 4 to 8 members, or R₂ and R₃ taken together form a substituted or unsubstituted aryl, heteroaryl, carbocyclyl, or heterocyclyl ring having 4 to 8 members;
formulated together with an ototoxic chemotherapeutic agent, either as a preparation or a kit, without diminishing the efficacy of the chemotherapeutic agent.
